(19) United States
(12) Patent Application Publication
(10) Pub. No.: US 2021/0398285 A1
(43) Pub. Date: Dec. 23, 2021

(54) CONSECUTIVE SLICE FINDING GROUPING

(71) Applicant: FOVIA, INC., Palo Alto, CA (US)

(72) Inventors: David WILKINS, Allison Park, PA (US); Kevin KREEGER, Palo Alto, CA (US)

(73) Assignee: FOVIA, INC., Palo Alto, CA (US)

(21) Appl. No.: 17/349,658

(22) Filed: Jun. 16, 2021

Related U.S. Application Data
(60) Provisional application No. 63/040,404, filed on Jun. 17, 2020.

(51) Int. Cl. G06T 7/00; G16H 30/40; G06N 20/00

(52) U.S. Cl. CPC G06T 7/0014; G06T 2207/20081; G06N 20/00; G16H 30/40

(57) ABSTRACT

A system and method for grouping objects (annotations or other marking) from multiple image slices into a single object, referred to as a grouped finding is provided, by grouping multiple slices as one single finding. Additionally, user interface interactions and controls are provided to efficiently navigate and interact with the grouped findings.

### CROSS-REFERENCE TO RELATED APPLICATIONS

This application claims the benefit of U.S. Provisional Patent Application No. 63/040,404, entitled, “CONSECUTIVE SLICE FINDING GROUPING,” filed Jun. 17, 2020, the content of which is incorporated herein by reference in its entirety for all purposes.

### FIELD

This relates generally to methods and systems for visualizing medical images and in one example, methods and systems for grouping consecutive image slices having findings.

### SUMMARY

According to one embodiment, a system and method for grouping objects (annotations or other marking) from multiple image slices into a single object, referred to as a grouped finding is provided, by grouping multiple slices as one single finding. Additionally, user interface interactions and controls are provided to efficiently navigate and interact with the grouped findings.

In one example, a computer-implemented method for grouping objects from multiple medical image slices of a set of medical images includes detecting objects from two or more slices of a set of medical images, determining if the detected objects are related, and associating the detected objects as a single finding in response to determining that the detected objects are related. Determining that the detected objects are related can be based on overlap in the x and y coordinate space when the two or more slices are overlapped. The method further includes forgoing associating the detected objects associating the detected objects if they are not determined related and/or are each found on single slice

The exemplary method further includes displaying the single finding, including the detected objects determined to be related, together for review. Further, the objects may be detected by an algorithm for identifying areas of interest in medical images (including, e.g., an artificial intelligence algorithm for identifying areas of interest in medical images or a machine learning algorithm for identifying areas of interest in medical images).

In other embodiments, a computer readable storage medium comprising instructions for carrying out the method and a system comprising a processor and memory having instructions for carrying out the method are provided.

### BRIEF DESCRIPTION OF THE DRAWINGS

FIGS. 1A and 1B illustrate exemplary image slices of a stack that are grouped into findings.

FIGS. 2A and 2B illustrate various process for grouping findings and analyzing a stack of images having grouped findings.

FIG. 3 illustrates an exemplary system for visualization of medical images

### DETAILED DESCRIPTION

There are many types of AI algorithms to assist radiologists in interpreting medical imaging studies. These include algorithms to assist in actual reading of the scanned images, algorithms to automatically find prior imaging studies of the patient, algorithms to make predictions based on other patient information than just the images, algorithms that help scheduling in the scanner rooms algorithms that assist in deciding what scans should be done, and many more. This patent is related to efficiently assisting the radiologist in reading the medical images.

The AI algorithms used to help detect or interpret disease and can be further subdivided into several categories. These include algorithms that classify disease, algorithms that measure structures in the images, algorithms that segment structures in the images, and many more.

This disclosure includes algorithms that detect or classify the disease in the images. Furthermore, this disclosure addresses algorithms commonly known as CAD (Computer Aided Detection), where the algorithm highlights multiple suspicious areas of abnormalities in the images.

In a radiology setting, it is advantageous to provide a mechanism that allows the user to more efficiently navigate the abnormalities, or findings, in the stack of images and quickly advanced through these findings to accept, reject, or modify each of these findings. Depending on the type of medical imaging modality, the multiple findings may not be visible all at once. The physician must scroll up and down through the image stack searching for the findings. It should be noted that a given study may have one or more stacks of images, where each stack may or may not have been processed by an AI algorithm.

There currently exists a standardized object that indicates key images in a stack of images such that the user can be quickly navigate between these important key images. Each finding contains one or more annotation markings per slice and may span multiple images in the stack.

When the physician reviews the findings, it is advantageous to efficiently navigate between each finding to accept or reject the entire finding with one action instead of a separate actions for each slice the finding intersects. An action could be from any number of input devices, such as a mouse, keyboard, gesture, voice command, user interface control on the screen, or any other way the user may interact with the system.

The concept of a key image works well when the user needs to navigate between any given image with some marking or annotation, this does not translate well for the concept of navigation between findings generated from an AI algorithm since many times these algorithms detect a finding that spans multiple images of the series (although not necessarily contiguous), or where multiple findings are on an individual image.

Some exemplary processes have the key image indicate the middle image in a set of the finding or indicate a key image for each image in the finding. These approaches are sub-optimal as neither case provides the object structure of how multiple images relate to a specific finding, and thus does not provide for efficient navigation since as user must manually navigate between neighboring slices. Also problematic is the issue when multiple findings are included within a single image, since it is generally not possible to distinguish between the two findings when performing the navigation.

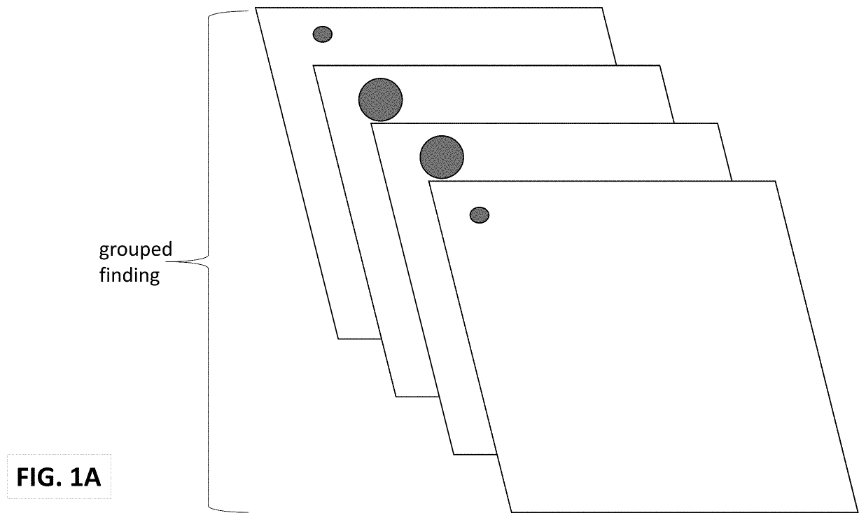

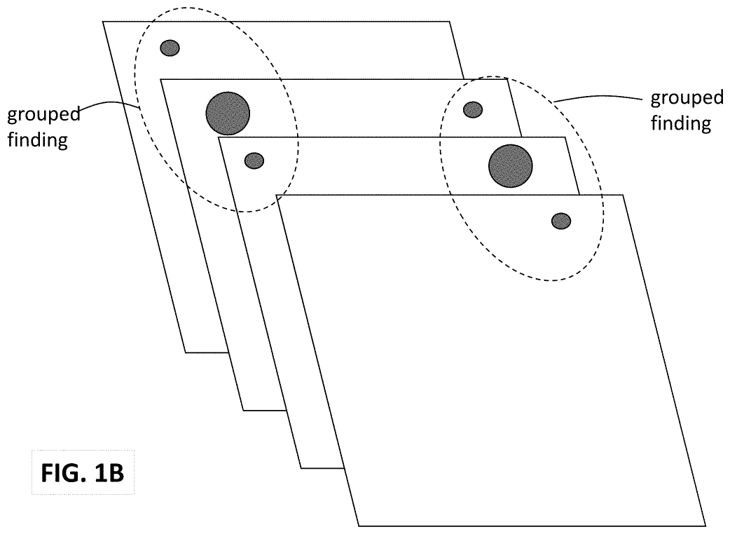

One embodiment of this invention provides a way to group objects (annotations or other marking) from multiple slices into a single object referred to as a grouped finding, by grouping multiple slices as one single finding (FIG. 1A), and also separating when multiple findings are on a single slice (FIG. 1B). Additionally, UI interactions and controls are provided to efficiently navigate and interact with grouped findings.

For example, FIG. 1A illustrates four consecutive image slices that include a finding that can be grouped as a single finding, which can be navigated to directly, e.g., to the first image or middle image within the single finding. Further, in FIG. 1B, the bottom three slices can be grouped as a first finding as indicated, and the top three slices as a second finding as indicated, where the two findings span across common images (e.g., the middle two images). Thus, when a user is finished with the first finding the user can navigate to the second finding that shares common image slices.

The grouping of objects across multiple slices can be determined or computed from a variety of approaches. For example, objects found on consecutive images that overlap in x and y coordinate space of the images can be grouped together as a single finding. There may be other heuristics that are incorporated that further refine how accurate the algorithm might be. For example, if the AI algorithm color codes each unique finding using a different color, if the color is available it can be used to ensure overlapping objects across different slices are correctly grouped together.

Organizing multiple objects across multiple slices allows the physician to accept/reject/modify each grouped finding as a single finding rather than a set of disparate pieces that each need to be reviewed independent of the other, thereby saving time and improving accuracy. It is important to note that this does not preclude the user from interacting with individual objects within the grouped finding for the case when the user does not agree with the grouping or wants to delete one or more objects from within the group.

One implementation of this uses various tags in a DICOM image to intelligently group these findings. This includes looking at elements of GSPS DICOM objects, SR (Structured Reports) DICOM objects, overlays in SC (secondary capture) DICOM objects, DICOM KOS (key image), DICOM DSO (segmentation object), vector overlay, heatmap overlays, segmentation objects and other objects created through AI algorithms.

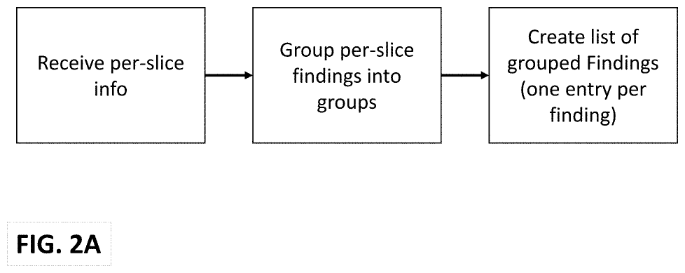

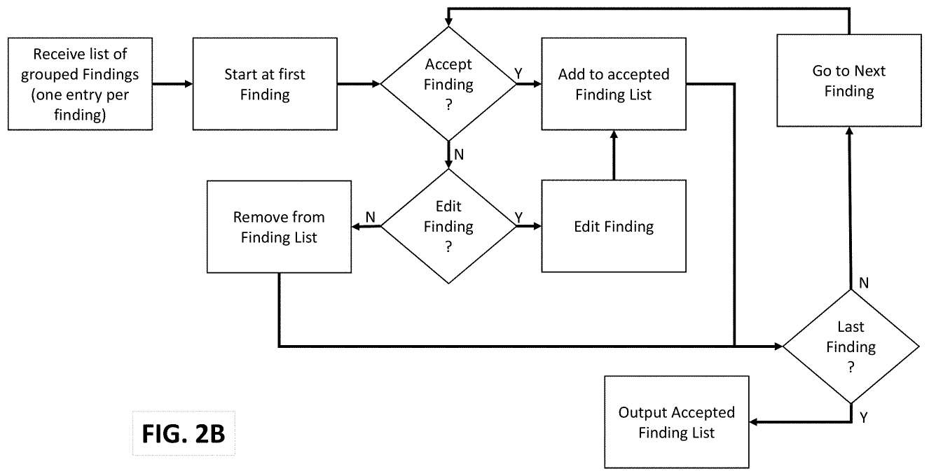

FIGS. 2A and 2B illustrate various process for grouping findings and analyzing a stack of images having grouped findings. With reference to FIG. 2A, a process for grouping findings is illustrated. Initially, a stack of images can be received, including information for each slice of the stack, e.g., including findings of areas of interest and the x and y coordinate of the areas of interest. The process may then group the per slice findings into groups, e.g., based on x and y coordinate overlap. The process may then create a list of findings, e.g., including the middle slice, first slice, first and last slice, and/or the like for of each grouped finding.

With reference to FIG. 2B, an example of reviewing a stack of medical images that have been processed to group findings is illustrated. Initially, a list of group findings is received or loaded and the system can load the first finding for review by a user, which may include viewing adjacent slices in the finding. The user can then accept the finding, edit the finding, or reject the finding. After accepting, editing, or rejecting the finding, the process can move to the next finding in the list of grouped findings. This process can repeat through the list of findings until all findings have been reviewed and can then output or generate a list of accepted findings.

Various embodiments described herein may be carried out by computer devices, medical imaging systems, and computer-readable medium comprising instructions for carrying out the described methods.

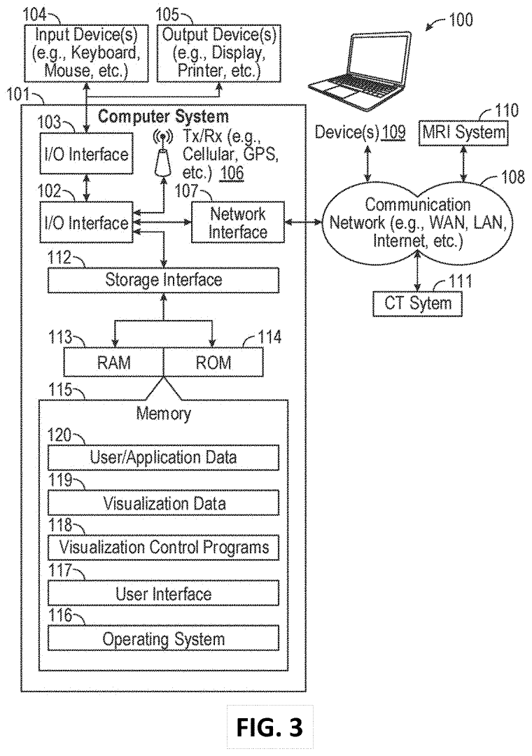

FIG. 3 illustrates an exemplary system 100 for visualization and analysis of medical images, consistent with some embodiments of the present disclosure. System 100 may include a computer system 101, input devices 104, output devices 105, devices 109, Magnet Resonance Imaging (MRI) system 110, and Computer Tomography (CT) system 111. It is appreciated that one or more components of system 100 can be separate systems or can be integrated systems. In some embodiments, computer system 101 may comprise one or more central processing units (“CPU” or “processor(s)”) 102. Processor(s) 102 may comprise at least one data processor for executing program components for executing user- or system-generated requests. A user may include a person, a person using a device such as those included in this disclosure, or such a device itself. The processor may include specialized processing units such as integrated system (bus) controllers, memory management control units, floating point units, graphics processing units, digital signal processing units, etc. The processor may include a microprocessor, such as AMD Athlon, Duron or Opteron, ARM's application, embedded or secure processors, IBM PowerPC, Intel's Core, Itanium, Xeon, Celeron or other line of processors, etc. The processor 102 may be implemented using mainframe, distributed processor, multi-core, parallel, grid, or other architectures. Some embodiments may utilize embedded technologies like application-specific integrated circuits (ASICs), digital signal processors (DSPs), Field Programmable Gate Arrays (FPGAs), etc.

Processor(s) 102 may be disposed in communication with one or more input/output (I/O) devices via I/O interface 203. I/O interface 103 may employ communication protocols/methods such as, without limitation, audio, analog, digital, monaural, RCA, stereo, IEEE-1394, serial bus, universal serial bus (USB), infrared, PS/2, BNC, coaxial, component, composite, digital visual interface (DVI), high-definition multimedia interface (HDMI), RF antennas, S-Video, VGA, IEEE 802.11 a/b/g/n/x, Bluetooth, cellular (e.g., code-division multiple access (CDMA), high-speed packet access (HSPA+), global system for mobile communications (GSM), long-term evolution (LTE), WiMax, or the like), etc.

Using I/O interface 103, computer system 101 may communicate with one or more I/O devices. For example, input device 104 may be an antenna, keyboard, mouse, joystick, (infrared) remote control, camera, card reader, fax machine, dongle, biometric reader, microphone, touch screen, touchpad, trackball, sensor (e.g., accelerometer, light sensor, GPS, gyroscope, proximity sensor, or the like), stylus, scanner, storage device, transceiver, video device/source, visors, electrical pointing devices, etc. Output device 105 may be a printer, fax machine, video display (e.g., cathode ray tube (CRT), liquid crystal display (LCD), light-emitting diode (LED), plasma, or the like), audio speaker, etc. In some embodiments, a transceiver 106 may be disposed in connection with the processor(s) 102. The transceiver may facilitate various types of wireless transmission or reception. For example, the transceiver may include an antenna operatively connected to a transceiver chip (e.g., Texas Instruments WiLink WL1283, Broadcom BCM4750IUB8, Infineon Technologies X-Gold 618-PMB9800, or the like), providing IEEE 802.11a/b/g/n, Bluetooth, FM, global positioning system (GPS), 2G/3G HSDPA/HSUPA communications, etc.

In some embodiments, processor(s) 102 may be disposed in communication with a communication network 108 via a network interface 107. Network interface 107 may communicate with communication network 108. Network interface 107 may employ connection protocols including, without limitation, direct connect, Ethernet (e.g., twisted pair 10/100/1000 Base T), transmission control protocol/internet protocol (TCP/IP), token ring, IEEE 802.11a/b/g/n/x, etc. Communication network 108 may include, without limitation, a direct interconnection, local area network (LAN), wide area network (WAN), wireless network (e.g., using Wireless Application Protocol), the Internet, etc. Using network interface 107 and communication network 108, computer system 101 may communicate with devices 109. These devices may include, without limitation, personal computer(s), server(s), fax machines, printers, scanners, various mobile devices such as cellular telephones, smartphones (e.g., Apple iPhone, Blackberry, Android-based phones, etc.), tablet computers, eBook readers (Amazon Kindle, Nook, etc.), laptop computers, notebooks, gaming consoles (Microsoft Xbox, Nintendo DS, Sony PlayStation, etc.), or the like. In some embodiments, computer system 101 may itself embody one or more of these devices.

In some embodiments, using network interface 107 and communication network 108, computer system 101 may communicate with MRI system 110, CT system 111, or any other medical imaging systems. Computer system 101 may communicate with these imaging systems to obtain images for display. Computer system 101 may also be integrated with these imaging systems.

In some embodiments, processor 102 may be disposed in communication with one or more memory devices (e.g., RAM 213, ROM 214, etc.) via a storage interface 112. The storage interface may connect to memory devices including, without limitation, memory drives, removable disc drives, etc., employing connection protocols such as serial advanced technology attachment (SATA), integrated drive electronics (IDE), IEEE-1394, universal serial bus (USB), fiber channel, small computer systems interface (SCSI), etc. The memory drives may further include a drum, magnetic disc drive, magneto-optical drive, optical drive, redundant array of independent discs (RAID), solid-state memory devices, flash devices, solid-state drives, etc.

The memory devices may store a collection of program or database components, including, without limitation, an operating system 116, user interface 117, medical visualization program 118, visualization data 119 (e.g., tie data, registration data, colorization, etc.), user/application data 120 (e.g., any data variables or data records discussed in this disclosure), etc. Operating system 116 may facilitate resource management and operation of computer system 101. Examples of operating systems include, without limitation, Apple Macintosh OS X, Unix, Unix-like system distributions (e.g., Berkeley Software Distribution (BSD), FreeBSD, NetBSD, OpenBSD, etc.), Linux distributions (e.g., Red Hat, Ubuntu, Kubuntu, etc.), IBM OS/2, Microsoft Windows (XP, Vista/7/8, etc.), Apple iOS, Google Android, Blackberry OS, or the like. User interface 117 may facilitate display, execution, interaction, manipulation, or operation of program components through textual or graphical facilities. For example, user interfaces may provide computer interaction interface elements on a display system operatively connected to computer system 101, such as cursors, icons, check boxes, menus, scrollers, windows, widgets, etc. Graphical user interfaces (GUIs) may be employed, including, without limitation, Apple Macintosh operating systems' Aqua, IBM OS/2, Microsoft Windows (e.g., Aero, Metro, etc.), Unix X-Windows, web interface libraries (e.g., ActiveX, Java, JavaScript, AJAX, HTML, Adobe Flash, etc.), or the like.

In some embodiments, computer system 101 may implement medical imaging visualization program 118 for controlling the manner of displaying medical scan images. In some embodiments, computer system 101 can implement medical visualization program 118 such that the plurality of images are displayed as described herein.

In some embodiments, computer system 101 may store user/application data 120, such as data, variables, and parameters (e.g., one or more parameters for controlling the displaying of images) as described herein. Such databases may be implemented as fault-tolerant, relational, scalable, secure databases such as Oracle or Sybase. Alternatively, such databases may be implemented using standardized data structures, such as an array, hash, linked list, struct, structured text file (e.g., XML), table, or as object-oriented databases (e.g., using ObjectStore, Poet, Zope, etc.). Such databases may be consolidated or distributed, sometimes among the various computer systems discussed above in this disclosure. It is to be understood that the structure and operation of any computer or database component may be combined, consolidated, or distributed in any working combination

It should be noted that, despite references to particular computing paradigms and software tools herein, the computer program instructions with which embodiments of the present subject matter may be implemented may correspond to any of a wide variety of programming languages, software tools and data formats, and be stored in any type of volatile or nonvolatile, non-transitory computer-readable storage medium or memory device, and may be executed according to a variety of computing models including, for example, a client/server model, a peer-to-peer model, on a stand-alone computing device, or according to a distributed computing model in which various of the functionalities may be effected or employed at different locations. In addition, references to particular algorithms herein are merely by way of examples. Suitable alternatives or those later developed known to those of skill in the art may be employed without departing from the scope of the subject matter in the present disclosure.

It will be understood by those skilled in the art that changes in the form and details of the implementations described herein may be made without departing from the scope of this disclosure. In addition, although various advantages, aspects, and objects have been described with reference to various implementations, the scope of this disclosure should not be limited by reference to such advantages, aspects, and objects. Rather, the scope of this disclosure should be determined with reference to the appended claims.

What is claimed is:

1. A computer-implemented method for grouping objects from multiple medical image slices of a set of medical images, the method comprising:
detecting objects from two or more slices of a set of medical images;
determining if the detected objects are related; and
associating the detected objects as a single finding in response to determining that the detected objects are related.

2. The method of claim 1, further comprising determining the detected objects are related based on overlap in the x and y coordinate space when the two or more slices are overlapped.

3. The method of claim 1, further comprising forgoing associating the detected objects if they are not determined related.

4. The method of claim 1, further comprising forgoing associating the detected objects if they are each found on single slice.

5. The method of claim 1, further comprising displaying the single finding, including the detected objects determined to be related, together for review.

6. The method of claim 1, wherein the objects are detected by a detection algorithm for identifying areas of interest in medical images.

7. The method of claim 6, wherein the stack of images was analyzed with an artificial intelligence algorithm for identifying areas of interest in medical images.

8. The method of claim 6, wherein the stack of images was analyzed with a machine learning algorithm for identifying areas of interest in medical images.

9. A computer readable storage medium, comprising instructions for:
detecting objects from two or more slices of a set of medical images;
determining if the detected objects are related; and
associating the detected objects as a single finding in response to determining that the detected objects are related.

10. The computer readable storage medium of claim 9, further comprising instructions for determining the detected objects are related based on overlap in the x and y coordinate space when the two or more slices are overlapped.

11. The computer readable storage medium of claim 9, further comprising instructions for forgoing associating the detected objects if they are not determined related.

12. The computer readable storage medium of claim 9, further comprising instructions for forgoing associating the detected objects if they are each found on single slice.

13. The computer readable storage medium of claim 9, further comprising instructions for displaying the single finding, including the detected objects determined to be related, together for review.

14. The computer readable storage medium of claim 9, wherein the objects are detected by a detection algorithm for identifying areas of interest in medical images.

15. The computer readable storage medium of claim 9, wherein the stack of images was analyzed with an artificial intelligence algorithm for identifying areas of interest in medical images.

16. The computer readable storage medium of claim 9, wherein the stack of images was analyzed with a machine learning algorithm for identifying areas of interest in medical images.

17. A system comprising a processor and memory, the memory storing instructions for:
detecting objects from two or more slices of a set of medical images;
determining if the detected objects are related; and
associating the detected objects as a single finding in response to determining that the detected objects are related.

18. The system of claim 17, further comprising instructions for determining the detected objects are related based on overlap in the x and y coordinate space when the two or more slices are overlapped.

19. The system of claim 17, further comprising instructions for forgoing associating the detected objects if they are not determined related.

20. The system of claim 17, further comprising instructions for forgoing associating the detected objects if they are each found on single slice.
